(19) United States
(12) Patent Application Publication
(10) Pub. No.: US 2017/0196475 A1
(43) Pub. Date: Jul. 13, 2017

(54) MAGNETIC RESONANCE IMAGING APPARATUS

(71) Applicant: HITACHI, LTD., Tokyo (JP)

(72) Inventor: Nobuyuki YOSHIZAWA, Tokyo (JP)

(73) Assignee: HITACHI, LTD., Tokyo (JP)

(21) Appl. No.: 15/326,263

(22) PCT Filed: Jul. 28, 2015
(86) PCT No.: PCT/JP2015/071336

§ 371 Date: Jan. 13, 2017

(30) Foreign Application Priority Data
Aug. 8, 2014 (JP) 2014-162909

(51) Int. Cl. A61B 5/055; A61B 5/00; G01R 33/563; A61B 5/026

(52) U.S. Cl. CPC A61B 5/055; A61B 5/026; A61B 5/742; G01R 33/56366

(57) ABSTRACT

In order to reduce inaccuracy of a hemodynamic visualization image acquired when a blood flow is labeled before blood flow visualization imaging, an MRI apparatus uses a blood flow velocity to control pulse sequences including a sequence of applying a high-frequency pulse for labeling the blood flow and imaging the subsequent blood flow or display of the hemodynamic visualization image. For example, the blood flow velocity is used for controlling application positions of one or more high-frequency pulses from among a plurality of the high-frequency pulses for labeling. The MRI apparatus controls time between labeling the blood flow and starting imaging and/or the application positions of high-frequency pulses for labeling the blood flow. A threshold value for color display of a blood flow visualization image is controlled.

### TECHNICAL FIELD

The present invention relates to a magnetic resonance imaging (hereinafter, referred to as “MRI”) apparatus and, in particular, to an MRI apparatus that performs blood flow imaging.

### BACKGROUND ART

An MRI apparatus is used for imaging to visualize a hemodynamic state with a difference between an image in which blood is labeled (a label image) and an image that is not labeled (a control image). In such imaging, blood vessel images and perfusion images can be acquired depending on the imaging timing. Thus acquired images are referred to as magnetic resonance (hereinafter, referred to as “MR”) perfusion images. The perfusion means a blood flow passing through the capillary circulation of an organ or a tissue region.

One of the imaging methods for MR perfusion images is an Arterial Spin Labeling method (hereinafter, referred to as “ASL”). In ASL, non-contrast imaging can generate the MR perfusion images.

As an example of ASL, the methods described in Patent Literatures 1 to 3 are taken.

PTL 1 describes that one 360-degree adiabatic labeling pulse is applied in order to acquire a label image and that two 180-degree adiabatic control pulses are applied in order to acquire a control image. PTL 2 describes that hundreds of high-frequency magnetic field (hereinafter, referred to as “RE”) pulses are applied in order to acquire a label or control image. PTL 3 discloses that two or three RF pulses are applied in order to acquire a label or control image.

### CITATION LIST

### Patent Literature

PTL 1: U.S. Patent Publication No. 5846197

PTL 2: U.S. Patent Publication No. 7545142

PTL 3: U.S. Patent Publication No. 6285900

### SUMMARY OF INVENTION

### Technical Problem

Although ASL has an advantage to be non-invasive because it does not use a contrast agent, there is a case where perfusion can be inaccurately evaluated in a hemodynamic visualization image acquired from ASL.

The causes of the above inaccurate evaluation are considered as follows.

1) When blood protons are labelled and controlled using a plurality of pulses, the blood protons move, and application positions of the second and subsequent RF pulses are shifted, which makes labeling and controlling imperfect.

2) A region of interest is imaged before the labeled and controlled blood protons are spread out in the region of interest or after the blood protons flow out of the region of interest.

3) Longitudinal relaxation of the labelled protons proceeds before the protons spread are spread out in a region of interest after label processing, which results in reducing labeling effects.

4) When a dynamic range of signal values is previously fixed during displaying a blood flow in a region of interest, the color display of hemodynamic images is inaccurate depending on the magnitude of an acquired signal value of a region of interest.

PTLs 1, 2, and 3 do not disclose the above problems as well as the solutions thereof.

The purpose of the present invention is to solve the above problems and reduce inaccuracy of a hemodynamic visualization image acquired from ASL. This acquires the hemodynamic visualization image in which an SNR (Signal-to-Noise Ratio) was improved or improves reliability of the hemodynamic visualization image displayed in color.

### Solution to Problem

In order to achieve the above purpose, the present invention uses a blood flow velocity to control a hemodynamic imaging pulse sequence accompanying a blood flow labeling process and a threshold value of color display of a hemodynamic image.

Specifically, the MRI apparatus of the present invention is characterized by comprising a static magnetic field generating magnet; a high-frequency magnetic field generation unit; a gradient magnetic field generation unit; a reception unit that receives nuclear magnetic resonance signals; and a control unit that controls the high-frequency magnetic field generation unit; the gradient magnetic field generation unit; and the reception unit according to a predetermined pulse sequence, that the pulse sequence includes sequences of applying a plurality of high-frequency pulses labeling blood flows (flowing bloods) and imaging the subsequent blood flows, and that the control unit uses a blood flow velocity to control application positions of one or more high-frequency pulses from among the plurality of high-frequency pulses.

Also, the MRI apparatus of the present invention is characterized by comprising a static magnetic field generating magnet; a high-frequency magnetic field generation unit; a gradient magnetic field generation unit; a reception unit that receives nuclear magnetic resonance signals; and a control unit that controls the high-frequency magnetic field generation unit; the gradient magnetic field generation unit; and the reception unit according to a predetermined pulse sequence, that the pulse sequence includes sequences of applying a high-frequency pulse labeling a blood flow (flowing blood) and imaging the subsequent blood flow, and that the control unit uses a blood flow velocity to control time between labeling the blood flow and starting the imaging and/or application positions of a high-frequency pulse for labeling the blood flow.

Also, the MRI apparatus of the present invention is characterized by comprising a static magnetic field generating magnet; a high-frequency magnetic field generation unit; a gradient magnetic field generation unit; a reception unit that receives nuclear magnetic resonance signals; a control unit that controls the high-frequency magnetic field generation unit; the gradient magnetic field generation unit; and the reception unit according to a predetermined pulse sequence; and a display/operation unit displaying a blood flow visualization image, that the pulse sequence includes sequences of applying a high-frequency pulse labeling a blood flow and imaging the subsequent blood flow, and that the display/operation unit is provided with a function of displaying in color based on a threshold value of signal strength of the blood flow visualization image to change the threshold value using a blood flow velocity.

### Advantageous Effects of Invention

The present invention can reduce inaccuracy of hemodynamic visualization images acquired from ASL. This can acquire a hemodynamic image whose SNR was improved or improve reliability of the hemodynamic image displayed in color.

### BRIEF DESCRIPTION OF DRAWINGS

FIG. 1 is a block diagram illustrating the overall configuration of an MRI apparatus to which the present invention is applied.

FIG. 2 is a schematic diagram of the procedure for displaying a hemodynamic image in color.

FIG. 3 is an explanatory diagram illustrating an example of a sequence used in a phase contrast method.

FIG. 4 is a graph of a blood flow velocity.

FIG. 5 is an explanatory diagram of an example of a labeling pulse or a control pulse.

FIG. 6 illustrates an example of main measurement to acquire a hemodynamic image.

FIG. 7 illustrates an example of a user interface.

FIG. 8 is a block diagram in which an entire control unit of a first embodiment is mainly illustrated.

FIG. 9 illustrates an example position of RF pulse application.

FIG. 10 illustrates a positional shift of RF pulse application caused by movement of blood protons.

FIG. 11 illustrates the procedure of the first embodiment.

FIG. 12 is a block diagram in which the entire control unit of second, third, and fifth embodiments is mainly illustrated.

FIG. 13 illustrates a relationship between a blood flow velocity and PLD.

FIG. 14 illustrates the procedure of the second embodiment.

FIG. 15 illustrates a relationship between a blood flow velocity and a label or control pulse application position.

FIG. 16 illustrates the procedure of the third embodiment.

FIG. 17 is a block diagram in which a display/operation unit of a fourth embodiment is mainly illustrated.

FIG. 18 illustrates examples of a blood flow velocity and a color bar.

FIG. 19 illustrates the procedure of the fourth embodiment.

FIG. 20 illustrates an example of a database.

FIG. 21 illustrates the procedure of the fifth embodiment.

### DESCRIPTION OF EMBODIMENTS

Hereinafter, the embodiments of the present invention will be described referring to the diagrams. It is noted that the same reference signs are used for the same functions in all the diagrams for describing the embodiments of the present invention, and the repeated descriptions are omitted.

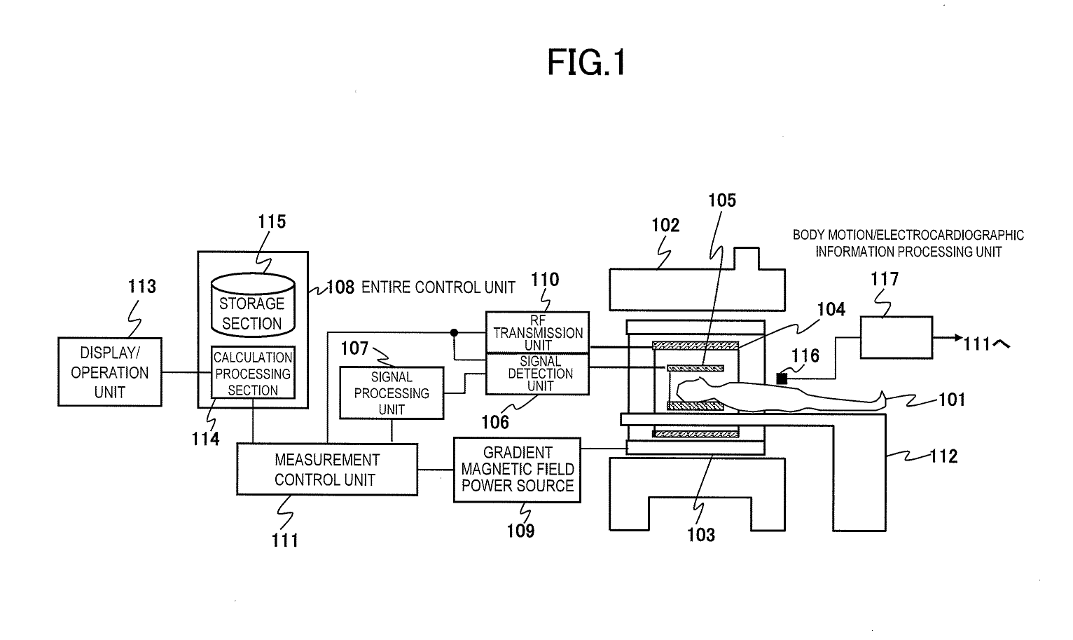

First, an example overview of an MRI apparatus to which the present invention is applied will be described based on FIG. 1.

FIG. 1 is block diagram illustrating an overall configuration of one embodiment of the MRI apparatus related to the present invention. The MRI apparatus acquires tomographic images of an object 101 using a nuclear magnetic resonance (hereinafter, referred to as “NMR”) phenomenon. As illustrated in FIG. 1, the MRI apparatus comprises a static magnetic field generating magnet 102, a gradient magnetic field coil 103 and a gradient magnetic field power source 109, a transmission RF coil 104 and an RF transmission unit 110, a reception RF coil 105 and a signal detection unit 106, a signal processing unit 107, a measurement control unit 111, an entire control unit 108, a display/operation unit 113, and a bed 112 that places the object 101 to carry the object 101 in/from the inside of the static magnetic field generating magnet 102.

The static magnetic field generating magnet 102 generates a homogeneous static magnetic field in each of an orthogonal direction to the body axis of the object 101 in case of the vertical magnetic field system and a body-axis direction in case of the horizontal magnetic field system, and a static magnetic field generation source of the permanent magnet system, the normal conduction system, or the superconducting system is disposed around the object 101.

The gradient magnetic field coil 103 consists of gradient magnetic field coils of the three-axis directions X, Y, and Z, each of the gradient magnetic field coils is connected to the gradient magnetic field power source 109 to drive the coils, and the electric current is supplied. Specifically, the gradient magnetic field power source 109 of each of the gradient magnetic field coils is driven by the command from the measurement control unit 111 to be described later, and the electric current is supplied to each of the gradient magnetic field coils. This generates gradient magnetic fields Gx, Gy, and Gz in the three-axis directions X, Y, and Z. According to the way of applying the gradient magnetic fields, an imaging cross section of an object is determined in order to apply phase encoding and frequency encoding to signals.

When a two-dimensional slice plane is imaged, a slice gradient magnetic field pulse (Gs) is applied in a direction orthogonal to the slice plane (imaging cross section) to set a slice plane for the object 101, and a phase encoding gradient magnetic field pulse (Gp) and a frequency encoding (lead-out) gradient magnetic field pulse (Gf) are applied in the remaining two directions orthogonal to the slice plane and orthogonal to each other to encode positional information in each direction for echo signals.

The transmission RF coil 104 is a coil irradiating RF pulses to the object 101 and is connected to the RF transmission unit 110 to supply a high-frequency pulse current. Hence, an NMR phenomenon is induced in atomic nucleus spin of atoms composing biological tissues of the object 101. Specifically, the RF transmission unit 110 is driven by the command from the measurement control unit 111 to be described later, high-frequency pulses are amplitude-modulated and supplied to the transmission RF coil 104 disposed in the vicinity of the object 101 after amplification, and then RF pulses are irradiated to the object 101.

The reception RF coil 105 is a coil receiving NMR signals (echo signals) to be emitted by the NMR phenomenon by the atomic nucleus spin of atoms composing biological tissues of the object 101 and is connected to the signal detection unit 106. The signal detection unit 106 performs a detection process for the echo signals received by the reception RF coil 105. Specifically, response echo signals of the object 101 induced by RF pulses irradiated from the transmission RF coil 104 are received by the reception RF coil 105 disposed in the vicinity of the object 101, and the signal detection unit 106 amplifies the received echo signals according to the command from the measurement control unit 111 to be described later, divides the signals into orthogonal two-system signals with quadrature phase detection, respectively samples the two-system signals by the predetermined number (such as 128, 256, and 512), converts each of the sampled signals into a digital amount with A/D conversion, and then transmits the converted signals to the signal processing unit 107 to be described. Therefore, the echo signals are acquired as time-series digital data (hereinafter, referred to as echo data) consisting of the predetermined number of sampling data.

The signal processing unit 107 performs various processes on the echo data and transmits the processed echo data to the measurement control unit 111.

The measurement control unit 111 mainly transmits various commands for data collection required to reconstruct tomographic images of the object 101 in order to control the gradient magnetic field power source 109, the RF transmission unit 110, and the signal detection unit 106. Specifically, the measurement control unit 111 operates under control of the entire control unit 108 to be described later, controls the gradient magnetic field power source 109, the RF transmission unit 110, and the signal detection unit 106 based on a predetermined pulse sequence, repeatedly executes application of RF pulses and gradient magnetic field pulses to the object 101and detection of echo signals from the object 101, and then collects echo data required to reconstruct images of an imaging region of the object 101.

As an example of a predetermined pulse sequence, given are pulse sequences for acquiring a blood flow velocity and MR perfusion images.

The entire control unit 108 controls the measurement control unit 111, various data processes, display and storage of the process results, and the like and comprises a calculation processing section 114 including a CPU and a memory inside and a storage section 115 such as an optical disk and a magnetic disk. Specifically, the measurement control unit 111 is controlled in order to execute echo data collection. When the echo data is input from the measurement control unit 111, the calculation processing section 114 stores the echo data in a region equivalent to k-space of the memory based on encoding information applied to the echo data. The echo data group stored in the region equivalent to k-space is referred to also as k-space data. Then, the calculation processing section 114 executes signal processing for the k-space data and processing such as image reconstruction by Fourier transform, displays images of the object 101 as the result on the display/operation unit 113 to be described later, and then records the images in the storage section 115.

In the present description, the measurement control unit 111 and the entire control unit 108 are collectively referred to also as the control unit.

The display/operation unit 113 comprises a display unit displaying reconstructed images of the object 101 and an operation unit such as a trackball, a mouse, and a key board inputting various control information of the MRI apparatus and control information about processes to be performed by the above entire control unit 108. The operation unit is disposed in the vicinity of the display unit and interactively controls various processes of the MRI apparatus through the operation unit while an operator is observing the display unit. The display unit has a color display function of a blood flow and can display images whose threshold values were changed for the color display.

Each part of the entire control unit 108 and each part of the display/operation unit 113 can consist of a CPU and a memory. In the memory, a program for executing each function is previously installed, and the CPU reads and executes the program in the memory. Consequently, operations of each part can be achieved. Although processing procedures of the entire control unit 108 and the display/operation unit 113 to be described later are described assuming that the processing procedures are realized as software, processes of the entire control unit 108 and the display/operation unit 113 can be realized by hardware such as ASIC and FPGA without limiting to the software in the present embodiments.

The MRI apparatus related to the present invention can be also provided with a body motion/electrocardiographic information detection unit that detects body motion/electrocardiographic information of an object as an external device. The body motion/electrocardiographic information detection unit comprises a sensor unit 116 that is mounted on the object 101 to detect the body motion/electrocardiographic information of the object and a body motion/electrocardiographic information processing unit 117 that processes signals from the sensor unit 116 and transmits the processed body motion/electrocardiographic information to the measurement control unit 111. The sensor unit 116 is a sensor for respiratory waveform detection in a case where the body motion/electrocardiographic information detection unit detects a respiratory waveform of the object, and the sensor unit 116 is an electrocardiograph, a pulse rate meter, or the like in a case where the body motion/electrocardiographic information detection unit detects electrocardiographic information of the object. The measurement control unit 111 executes a pulse sequence by synchronizing with the body motion/electrocardiographic information of the object detected by the body motion/electrocardiographic information detection unit (synchronous imaging).

In FIG. 1, the transmission RF coil 104 and the gradient magnetic field coil 103 on the transmission side are installed, for example, so as to be opposite to the object 101 in case of the vertical magnetic field system or so as to surround the object 101 in case of the horizontal magnetic field system in the static magnetic field space of the static magnetic field generating magnet 102 in which the object 101 is carried. The reception RF coil 105 on the reception side is also installed so as to be opposite to or so as to surround the object 101.

Next, the operation procedure of the control unit will be mainly described based on the overall configuration of the above embodiment.

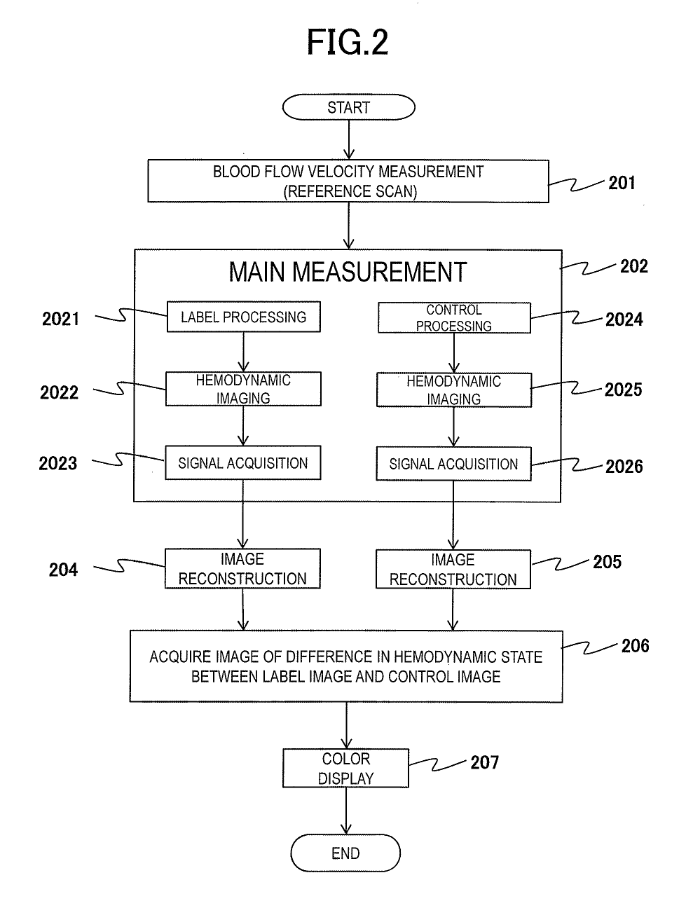

FIG. 2 illustrates the overview of the operation procedure of the present embodiments.

The control unit controls blood flow velocity measurement (reference scan) 201 for obtaining a blood flow velocity. The blood flow velocity obtained by the reference scan 201 is used for sequence control of main measurement 202 in the entire control unit 108 and color display 207 in the display/operation unit 113.

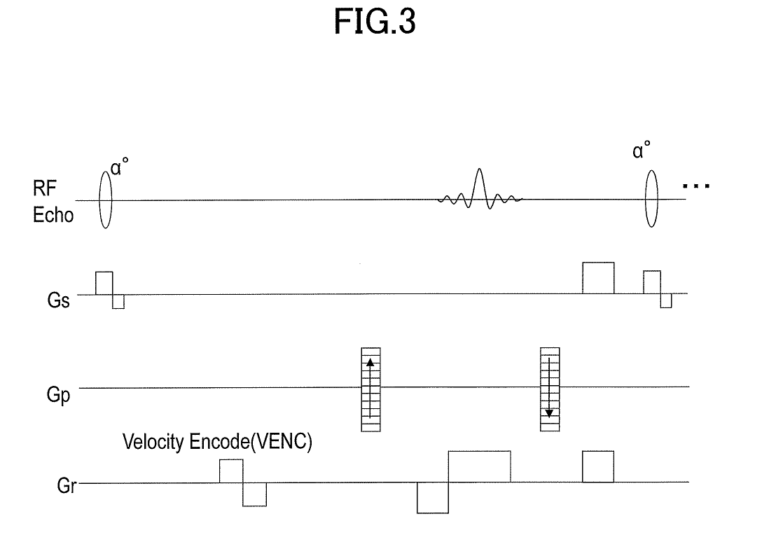

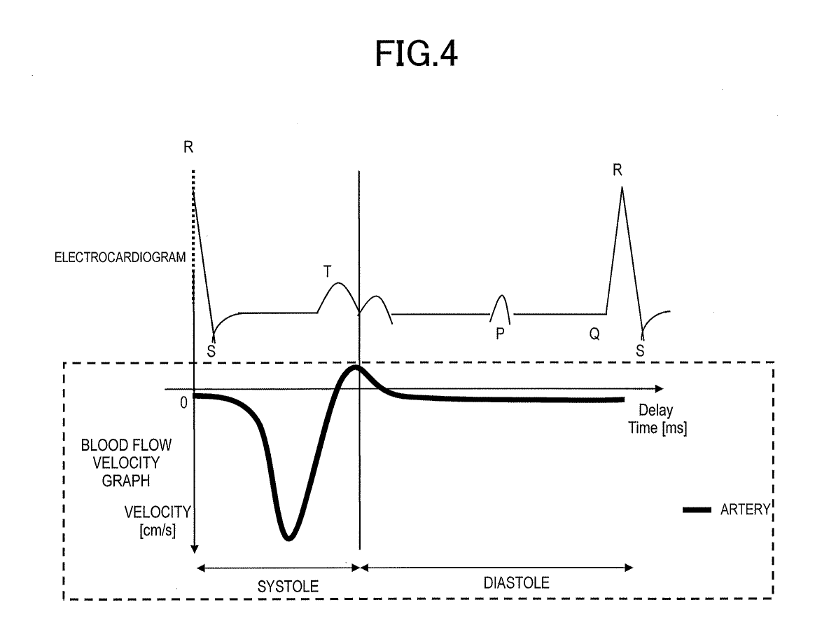

For example, a pulse sequence of a PC (Phase Contrast) method excellent in blood flow velocity visualization can be used for the reference scan 201 for obtaining a blood flow velocity. FIG. 3 illustrates an example of a sequence diagram of the PC method using a flow encode pulse that provides a phase shift proportional to a blood flow velocity. FIG. 4 illustrates an example of a blood flow velocity graph acquired using the PC method. An average blood flow velocity of each object can be evaluated from the acquired blood flow velocity graph. Alternatively, in case of performing synchronous imaging, not the average blood flow velocity but a blood flow velocity at a desired Delay Time (delay time from the R wave) may be evaluated.

Not only the MRI apparatus but also the other devices can be used for the blood flow velocity measurement. A blood flow velocity may be obtained by storing general relationships between object information such as the height, age, weight, and sex of an object and the blood flow velocity in a database and accessing the database when a user inputs the object information instead of the blood flow velocity measurement.

Next, the control unit controls the main measurement 202 to acquire hemodynamic images. The main measurement 202 includes label processing 2021 and control processing 2024 as well as hemodynamic imaging 2022 and hemodynamic imaging 2025 to be performed after those processes. Although the hemodynamic images include both perfusion images and blood vessel images, the perfusion images will be mainly described as an example in the following description.

A known method capable of acquiring hemodynamic images in a non-contrast manner may be used for the main measurement 202. An ASL (Arterial Spin Labeling) method is known as such a known method. Additionally, PASL (Pulsed Arterial Spin Labeling), CASL (Continuous Arterial Spin Labeling), pCASL (Pseudo-Continuous Arterial Spin Labeling), and the like are given as specific examples of the ASL method.

The label processing 2021 reverses the spin of a selected site, and the control processing 2024 makes longitudinal magnetization 0 degree of the spin of a selected site. One or more pulses of high-frequency pulses are applied in the label processing 2021 or the control processing 2024. A known method may be used in the label processing 2021 or the control processing 2024. A label/control RF pulse processing to be used in a known method capable of acquiring perfusion images in the above non-contrast manner is given as a known method.

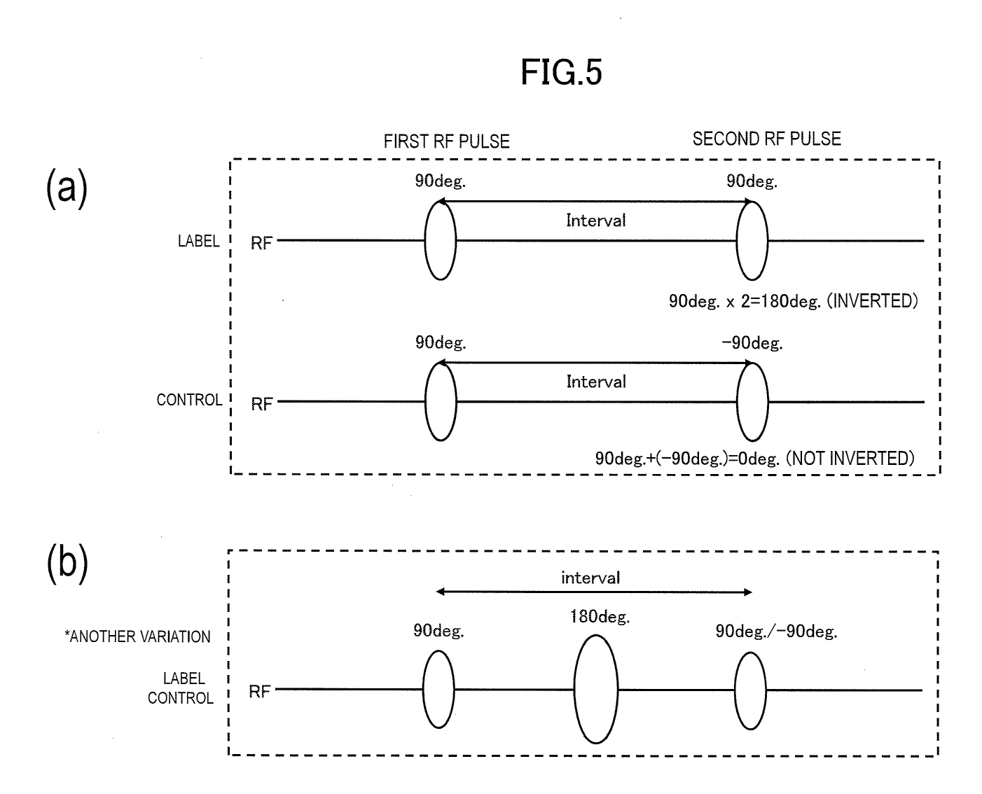

FIG. 5 illustrates examples of RF pulses for the label processing 2021 and RF pulses for the control processing 2024. FIG. 5(a) illustrates examples of using two of 90-degree pulses as labeling RF pulses as well as using a 90-degree pulse and a −90-degree pulse as controlling RF pulse, and FIG. 5(b) illustrates an example of using a 90-degree pulse, a 180-degree pulse, and a 90-degree pulse as the labeling RF pulses and a 90-degree pulse, a 180-degree pulse, and a −90-degree pulse as controlling RF pulses. Here, the 180-degree pulse is a pulse for re-focusing protons on which lateral magnetization was performed with the 90-degree pulse and phase dispersion was performed due to static magnetic field (B0) inhomogeneity (re-focusing pulse). It is noted that a flip angle and the number of applications of the RF pulses are not limited to the above.

A known imaging method capable of acquiring blood vessel images or perfusion images can be adopted for hemodynamic imaging 2022 or 2025. The known imaging method includes a spin echo-type echo planer method (SE-EPI), a fast spin echo method (FSE), a gradient echo-type echo planer method (GE-EPI), and the like.

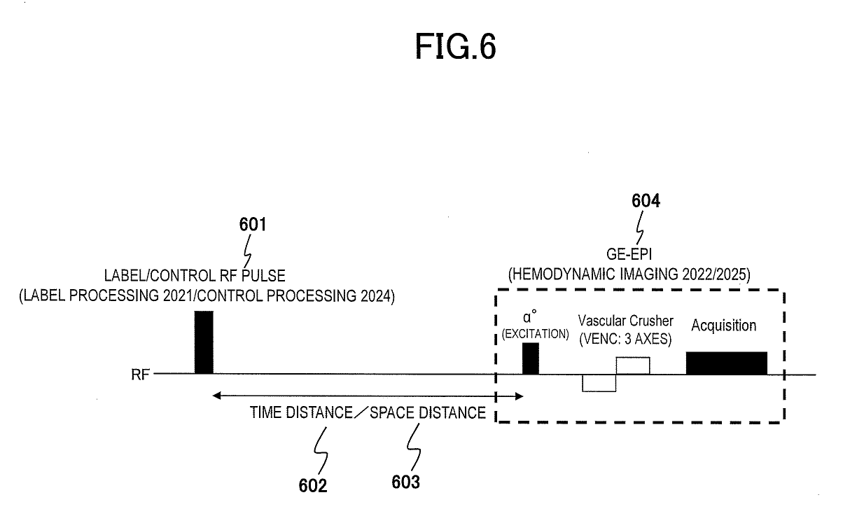

Referring to FIG. 6, described is a relationship between the sites for the label processing 2021 and the control processing 2024 of FIG. 2 and the sites for both the hemodynamic imaging 2022 and 2025. Although the label processing and the control processing are different, they are illustrated together in order to simplify the description in the diagram.

The site for performing the label processing 2021 or the control processing 2024 is set to a predetermined position on the upper flow side of a blood flow compared to a target site at which the hemodynamic imaging 2022 or 2025 is performed by considering a velocity of the blood flowing from the site to the target site at which the hemodynamic imaging 2022 or 2025 is performed and time distance 602/space distance 603 between performing the label processing 2021 or the control processing 2024 and performing the hemodynamic imaging 2022 or 2025.

Here, the time distance 602 is time between performing the label processing 2021 or the control processing 2024 and starting the hemodynamic imaging 2022 or 2025. The space distance 603 is a distance between a position of the hemodynamic imaging 2022 or 2025 and a position for applying label/control processing RF pulses.

In case of performing label or control processing on a blood flow using a plurality of RF pulses, the time distance 602 may be time between first or last processing and starting imaging, and the space distance 603 may be a distance between a position of hemodynamic imaging and a first or last RF pulse application position.

The time distance 602 can be referred to as PLD (Post Label Delay). It is desired that imaging a region of interest is performed in time when label- or control-processed blood protons reach the region of interest, and the PLD is desirably time until the label- or control-processed blood is spread all over the region of interest. On the other hand, the PLD should be set shorter as possible so that longitudinal relaxation does not proceed because the longitudinal relaxation proceeds in a case where the PLD is too long. It is clinically desirable to perform imaging at a plurality of time distances (PLD) because optimal PLD differs depending on the object.

In the main measurement 202, a site to perform the label processing 2021 is selected, a blood flow is labeled by applying RF pulses for labeling a blood flow, and then a pulse sequence for the hemodynamic imaging 2022 is executed by selecting a target site in a position separated by a predetermined time distance 602/space distance 603 in order to acquire signals required for image reconstruction (2023 of FIG. 2).

Next, a site to perform the control processing 2024 is selected to apply RF pulses for control processing, the same target site is selected in a position separated by a predetermined time distance 602/space distance 603, and then the same pulse sequence of the hemodynamic imaging 2025 is executed in order to acquire signals (2026). Images visualizing hemodynamics are acquired (206) by taking a difference between an image reconstructed from the signals acquired after the label processing (204) and an image reconstructed from the signals acquired after the control processing (205).

Either of the label processing 2021 or the control processing 2024 may be first performed, and signals required for image reconstruction may be finally acquired by alternately performing label processing to signal acquisition (2021 to 2023) and control processing to signal acquisition (2024 to 2026).

The acquired image, i.e. a perfusion image is displayed in color (207). The color display is performed by assigning colors to signal strength according to predetermined threshold values. A color bar is also displayed to indicate the threshold values of the color image display.

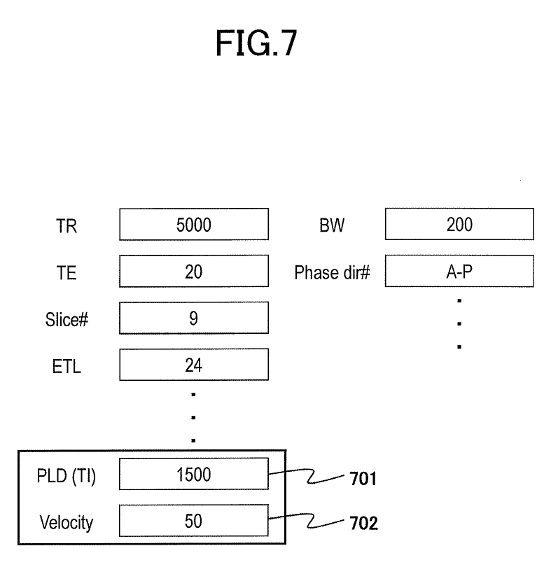

It is noted that scanning conditions and scan parameters required for main measurement can be input from a user interface (UI) as illustrated in FIG. 7 by a user. Information such as a blood flow velocity, PLD, a space distance (not illustrated in the diagram), and an application position of a high-frequency pulse (not illustrated in the diagram) for labeling/controlling is simultaneously input as needed.

As described above, referring to FIG. 2 and the like, the operational overview of the MRI apparatus of the present embodiments are described. The present embodiments reflect a result acquired in the blood flow velocity measurement 201 to the main measurement 202 and/or the color display 207 to be performed next and improve the accuracy to visualize perfusion, which can adopt several forms as utility forms of a blood flow velocity. Hereinafter, each embodiment having a different utility form will be described.

### Embodiment 1

An MRI apparatus of Embodiment 1 is characterized by that a control unit uses a blood flow velocity in order to control application positions of high-frequency pulses after the second pulse of a plurality of the high-frequency pulses. In Embodiment 1, a blood flow velocity in a label or control region is acquired in the blood flow velocity measurement (reference scan) 201. The sequence of the main measurement 202 is controlled using the acquired blood flow velocity in order to make the RF pulse application positions to track the blood flow in the label processing 2021 or the control processing 2024. Specifically, the blood flow velocity is used for controlling RF pulse application positions after the second RF pulse labeling the blood flow.

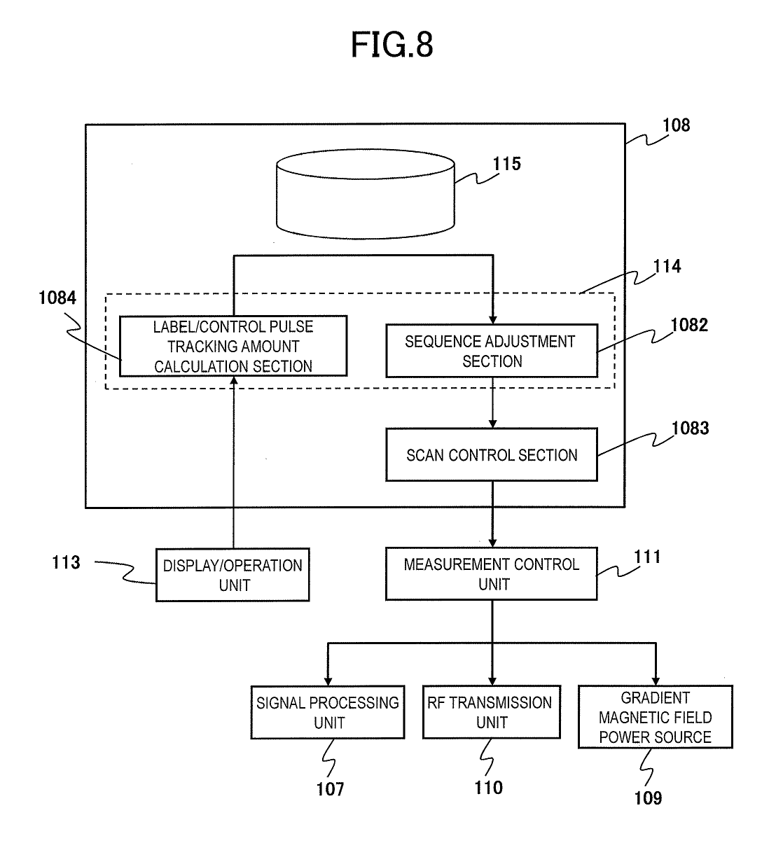

FIG. 8 is a functional block diagram in which the entire control unit 108 of the present embodiment is mainly illustrated. In FIG. 8, the same functions as FIG. 1 are indicated with the same reference signs, and the descriptions thereof are omitted.

As illustrated, the entire control unit 108 includes a label/control pulse tracking amount calculation section 1081 and a sequence adjustment section 1082.

The label/control pulse tracking amount calculation section 1081 calculates a change amount (tracking amount) in label or control positions using a blood flow velocity in a label or control processing region as described later.

The sequence adjustment section 1082 adjusts a main measurement sequence based on the calculation result acquired by the label/control pulse tracking amount calculation section 1081. Specifically, the sequence adjustment section 1082 adjusts at least either of a frequency or a gradient magnetic field application amount of RF pulses based on the acquired tracking amount in order to adjust application positions of the RF pulses.

A scan control section 1083 controls operations such as start and stop of scanning.

Described will be calculation to be performed by the label/control pulse tracking amount calculation section 1081.

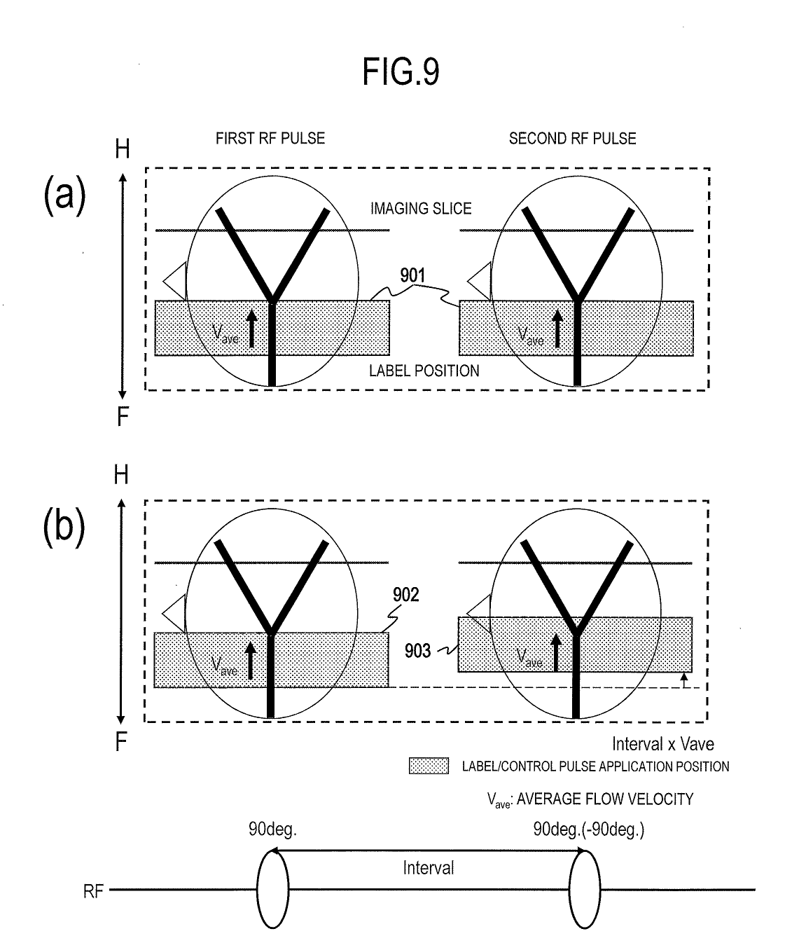

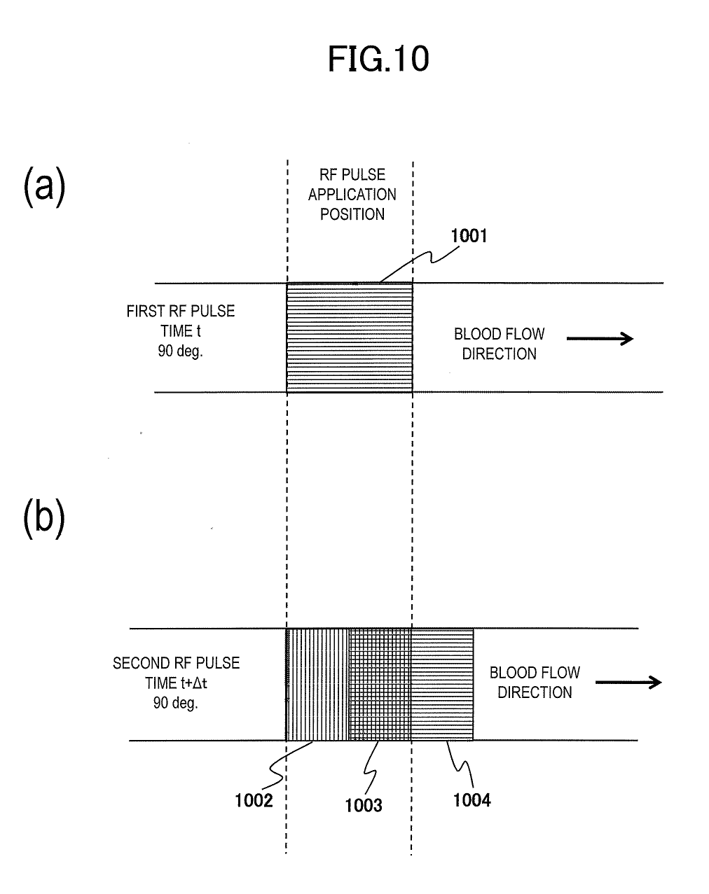

First, referring to FIGS. 9 and 10, description will be made for a case where RF pulse application positions are shifted depending on the blood flow velocity. Here, as an example, described will be a case of using two RF pulses as labeling/controlling RF pulses.

The labeling RF pulses and the control processing RF pulses comprises the same number of multiple RF pulses, and the pulse intervals are set as short as possible in order to avoid B0 inhomogeneity effects (refer to FIG. 5).

However, even if the pulse intervals are short, the blood flow moves in the meantime. Therefore, in case of selecting the same site 901 and performing label/control processing like a conventional technique illustrated in FIG. 9(a), labeling or controlling becomes incomplete because the label/control processing is not performed on the actually same blood.

Using FIG. 10, the incompleteness of the label processing will be described specifically. FIG. 10 illustrates an example in case of using two 90-degree pulses as RF pulses. First, FIG. 10(a) illustrates application of the first 90-degree RF pulse. 1001 of the diagram is a region selected by the RF pulse application.

Next, FIG. 10(b) illustrates RF pulse application of the second 90-degree RF pulse after Δt hours. Because blood protons on the region 1001 move according to a blood flow velocity at this time, only a region 1003 is a region where spin is reversed (label-processed) by the second 90-degree RF pulse. It is noted that a region 1004 is a region on which the first RF pulse processing has been performed and the second RF pulse processing is not performed and that a region 1002 on which the first RF pulse processing is not performed and the second RF pulse processing has been performed on the other hand.

The present embodiment reduces the incompleteness label/control processing that depends on a blood flow speed by tracking a blood flow velocity to move a site on which label/control processing is performed.

FIG. 9(b) indicates application positions of RF pulses for the label/control processing of the present embodiment. FIG. 9 illustrates an example of using two 90-degree pulses as the RF pulses for the label/control processing. In the diagram, H stands for Head, and F stands for Foot. The present embodiment allows an application position 903 of the second RF pulse to track a blood flow according to a blood flow velocity of each object after RF pulses of the label or control processing are applied in a position 902.

Hence, the label/control pulse tracking amount calculation section 1081 calculates a label or control pulse tracking amount as follows.

Vₐᵥₑ stands for an average value of the blood flow velocity evaluated by the reference scan, and Interval stands for an RF pulse interval as follows.

Δd=Vₐᵥₑ×Interval [Equation 1]

The tracking amount Δd is calculated using the above equation.

In a case where the label/control RF pulses comprise three or more pulses (n pulses), an application position tracking amount may be calculated by Δd in the last RF pulse application position or may be calculated using the following equation in the first RF pulse application position.

Δdi=Vₐᵥₑ×Interval×(i−1)_{i=3,4, . . . n} [Equation 2]

In a case where the label/control RF pulses comprise n pulses, application positions are tracked by m pulses (n>=m) according to a blood flow velocity and a label thickness, and it is configured that tracking from the first to m-th pulses is repeated.

A tracking amount may be also calculated from a blood flow velocity in desired Delay Time (delay time from the R-wave) in case of synchronous imaging.

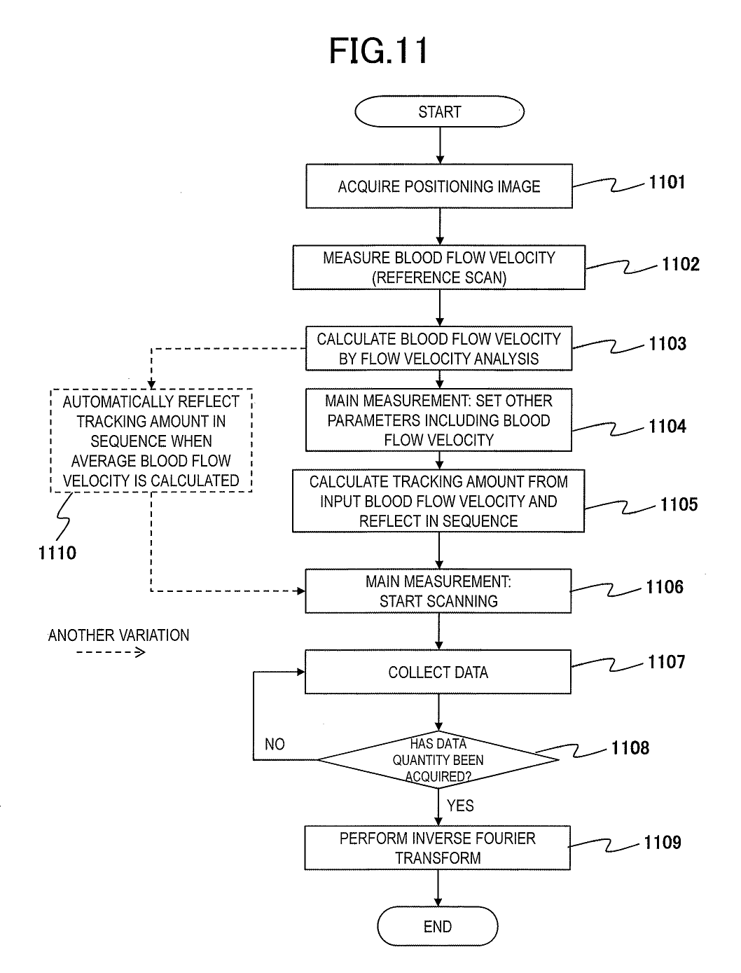

The procedure for the MRI apparatus and the control unit of the present embodiment will be described using FIG. 11.

A positioning image is imaged in order to set an imaging position (Step 1101).

Using the positioning image imaged in Step 1101, blood flow velocity measurement (reference scan) is performed on a region specified by an operator (Step 1102).

A blood flow velocity graph is evaluated by a flow velocity analysis based on data acquired the reference scan in order to calculate a blood flow velocity (Step 1103).

A display/operation unit 113 is used for setting scan parameters input for main measurement. At this time, the blood flow velocity evaluated in Step 1103 is also input as a scan parameter (Step 1104).

The label/control pulse tracking amount calculation section 1081 calculates a tracking amount of label or control pulses according to the equation (1) or (2) using the input blood flow velocity. The sequence adjustment section 1082 adjusts a sequence based on the calculation result (Step 1105).

Inputting the start button starts scanning for main measurement (Step 1106). That is, performed are label processing 2021, hemodynamic imaging 2022, control processing 2024, hemodynamic imaging 2025, and the like of FIG. 2.

The measurement control unit 111 collects data (Step 1107).

The measurement control unit 111 judges whether or not a predetermined amount of data determined by parameters set by an operator in Step 1104 has been acquired and proceeds to Step 1107 in a case where the acquisition has not been completed or proceeds to Step 1109 in a case where the acquisition has been completed (Step 1108).

The calculation processing section 114 reconstructs two-dimensional or three-dimensional images by performing a Fourier transform on k-space data (Step 1109).

As illustrated in the dashed-line arrow of FIG. 11 as a variation example, it may be configured so that a tracking amount is automatically reflected to a sequence when a blood flow velocity is calculated in Step 1103 in order to start main measurement of Step 1106 (Step 1110).

The present embodiment reflects a blood flow velocity to the main measurement sequence and makes RF pulse application positions for performing label or control processing track a blood flow, which can perform the label or control processing on the blood flow more efficiently than conventional methods. Consequently, highly reliable hemodynamic images in which the SNR was improved can be acquired.

### Embodiment 2

An MRI apparatus of Embodiment 2 is characterized by that the control unit controls time from labeling a blood flow to starting imaging using a blood flow velocity. That is, although Embodiment 1 makes RF pulse application positions for performing label or control processing track a blood flow, Embodiment 2 differently uses the blood flow velocity for adjusting time from label/control processing to starting imaging (PLD) in main measurement. It is noted that the blood flow velocity includes a blood flow velocity from a label or control processing region to an imaging region but is not limited to this.

In a case where a distance from an imaging position to a position to which label or control RF pulses are applied is fixed, for example, it takes a longer time to reach a region of interest than a case of a high blood flow velocity in case of a low blood flow velocity.

It is clinically desirable to perform imaging at a plurality of time distances (PLDs) because time until labeled or controlled blood protons to reach a region of interest differs depending on the object. On the other hand, imaging at the plurality of PLDs results in extending imaging time, and there are some cases where such imaging is not accepted in the view point of examination time.

The present embodiment performs imaging at a PLD suitable for an object using a blood flow velocity obtained by a reference scan in order to acquire highly reliable hemodynamic images by performing imaging only once. That is, the start of imaging a blood flow is controlled by using the blood flow velocity to adjust the PLDs. It is noted that the PLD is time from applying the last RF pulse from among a plurality of label or control RF pulses to starting imaging in the present embodiment.

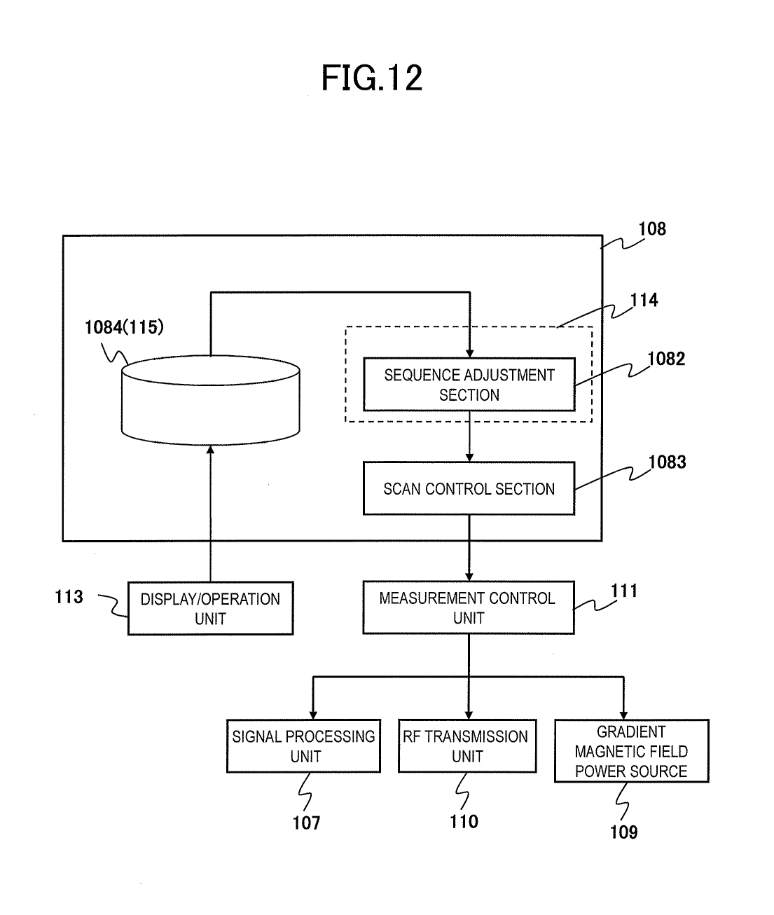

FIG. 12 is a functional block diagram in which the entire control unit 108 of the present embodiment is mainly illustrated. In FIG. 12, the same functions as FIG. 1 are indicated with the same reference signs, and the descriptions thereof are omitted. The entire control unit 108 is characterized by comprising a database 1084 and the sequence adjustment section 1082.

The entire control unit 108 accesses the database 1084 to acquire a PLD suitable for a blood flow velocity obtained a reference scan.

The database 1084 includes data of a relationship between a blood flow velocity and a PLD in the brain based on a standard model of humans. The equation (3) shows an example of the relationship between a blood flow velocity and a PLD.

$${PLD}_{A} = \frac{d 1}{{V}_{ave 1}} + \frac{d 2}{sin θ \times {V}_{ave 2}} + α \qquad [ Equation 3 ]$$

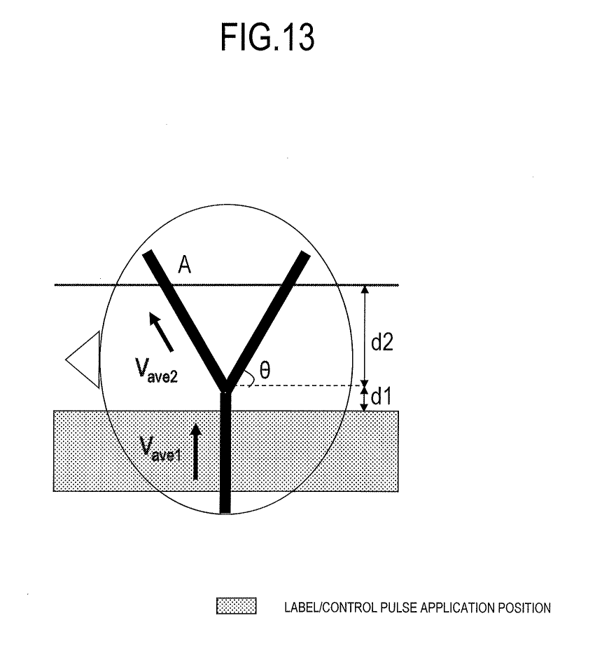

As illustrated to FIG. 13, the equation (3) represents time until labeled or controlled blood reaches the point A in an imaging slice. However, FIG. 13 simplifies blood vessel running in the brain for easy understanding. In the diagram, Vₐᵥₑ₁ is an average blood flow velocity until the blood reaches a branch point of the blood vessel, Vₐᵥₑ₂ is an average blood flow velocity from the branch point of the blood vessel to A in the imaging slice, d1 is a distance from an application position of a label or control RF pulse to the branch point, and d2 is a distance from the branch point to A in the imaging slice. θ is a branching angle. Also, a indicates a fluctuation of each living body until perfusion signals are shown after labeled blood reaches a perfusion region. Equation (3) and FIG. 13 shows that a suitable PLD and a blood flow velocity are closely related to each other.

Such a relationship between the suitable PLD and the blood flow velocity is stored in the database 1084.

The sequence adjustment section 1082 reflects a suitable PLD acquired from the above relational equation between the PLD and the blood flow velocity to a sequence.

The scan control section 1083 controls operations such as start and stop of scanning.

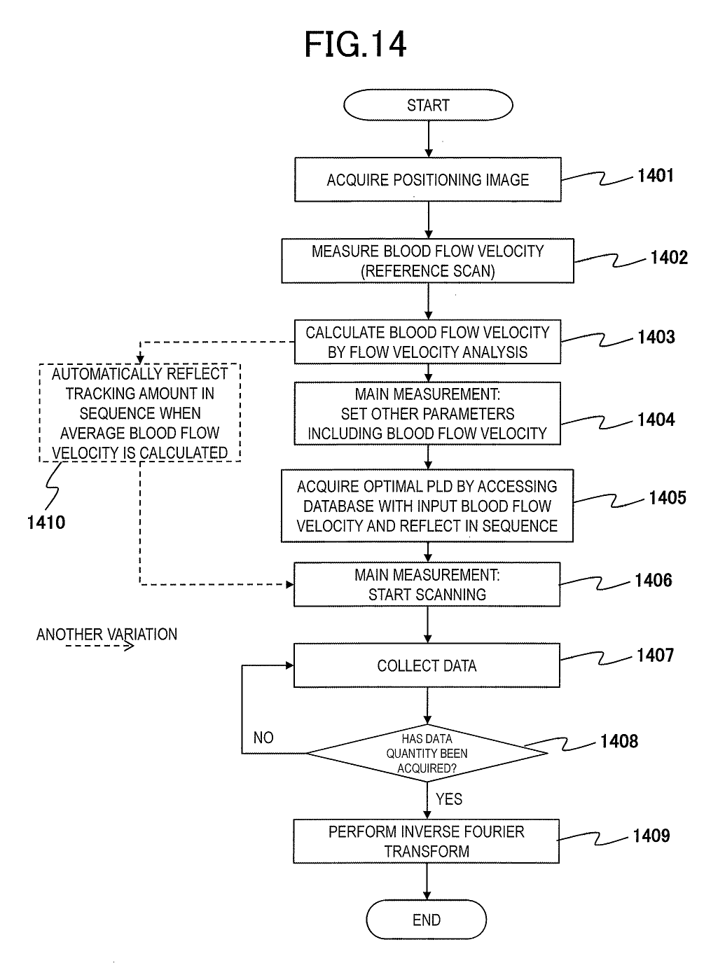

The procedure for the MRI apparatus and the control unit of the present embodiment will be described using FIG. 14.

A positioning image for setting a imaging position is imaged (Step 1401).

Using the positioning image imaged in Step 1401, the blood flow velocity measurement (reference scan) is performed on a region specified by an operator (Step 1402).

A blood flow velocity graph is evaluated by the flow velocity analysis based on data acquired the reference scan in order to calculate a blood flow velocity (Step 1403).

The display/operation unit 113 is used for setting scan parameters input for main measurement. At this time, the blood flow velocity evaluated in Step 1403 is also input as a scan parameter (Step 1404).

The entire control unit 108 accesses the database 1084 to acquire a PLD suitable for the input blood flow velocity. The sequence adjustment section 1082 reflects the suitable PLD to a sequence (Step 1405).

Pressing the start button starts scanning for main measurement (Step 1406). That is, performed are the label processing 2021, the hemodynamic imaging 2022, the control processing 2024, the hemodynamic imaging 2025, and the like of FIG. 2.

The measurement control unit 111 collects data (Step 1407).

The measurement control unit 111 judges whether or not a predetermined amount of data determined by parameters set by an operator in Step 1404 has been acquired and proceeds to Step 1407 in a case where the acquisition has not been completed or proceeds to Step 1409 in a case where the acquisition has been completed (Step 1408).

The calculation processing section 114 reconstructs two-dimensional or three-dimensional images by performing a Fourier transform on k-space data (Step 1409).

As illustrated in the dashed-line arrow of FIG. 14 as a variation example, it may be configured so that a suitable PLD is automatically reflected to a sequence when a blood flow velocity is calculated in Step 1403 in order to start the scanning for main measurement of Step 1406 (Step 1410).

The present embodiment performs imaging at a PLD suitable for each object using a blood flow velocity of the object, which can acquire highly reliable hemodynamic images without being affected by the blood flow velocity by performing imaging only once.

### Embodiment 3

An MRI apparatus of Embodiment 3 is characterized a control unit uses a blood flow velocity in order to control application positions of high-frequency pulses for labeling a blood flow. That is, although Embodiment 2 is a case of changing a PLD according to the blood flow velocity, Embodiment 3 uses the blood flow velocity in order to adjust application positions of label or control processing RF pulses (the space distance 603 of FIG. 6) differently from Embodiment 2.

It is noted that the blood flow velocity includes a blood flow velocity from a label or control processing region to an imaging region but is not limited to this.

In a case where the PLD is too long, longitudinal relaxation of label-processed blood protons proceeds, which results in reducing labeling effects. On the other hand, in case of setting the PLD shorter in light of the longitudinal relaxation, there can be a case where blood protons with a low flow velocity have not reached a region of interest.

The present invention performs imaging at an optimal space distance using a blood flow velocity obtained by a reference scan, which acquires highly reliable hemodynamic images in which the SNR was improved without prolonging a PLD (by preventing labeling effect reduction due to longitudinal relaxation).

Specifically, the blood flow velocity obtained by a reference scan is used for controlling positions to which label or control RF pulses are applied (hereinafter, referred to as “label or control positions”).

FIG. 12 is a functional block diagram in which the entire control unit 108 of the present embodiment is mainly illustrated. In FIG. 12, the same functions as FIG. 1 are indicated with the same reference signs, and the descriptions thereof are omitted. The entire control unit 108 is characterized by including the database 1084 and the sequence adjustment section 1082.

The database 1084 includes data related to a relationship between a blood flow velocity and optimal label or control positions based on a standard model of humans. Equations (4) and (5) show examples of relationships between the blood flow velocity and the optimal label or control positions. It is noted that a described in Equations (4) and (5) is similar to a described in Equation (3).

$$\begin{matrix}PLD 1000 [ ms ] = \frac{d 1}{{V}_{ave 1}} + \frac{d 2}{sin θ \times {V}_{ave 2}} + α & [ Equation 4 ] \\ PLD 1000 [ ms ] = \frac{{d 1}^{′}}{{V}_{ave 1}^{′}} + \frac{{d 2}^{′}}{sin {θ}^{′} \times {V}_{ave 2}^{′}} + α & [ Equation 5 ]\end{matrix}$$

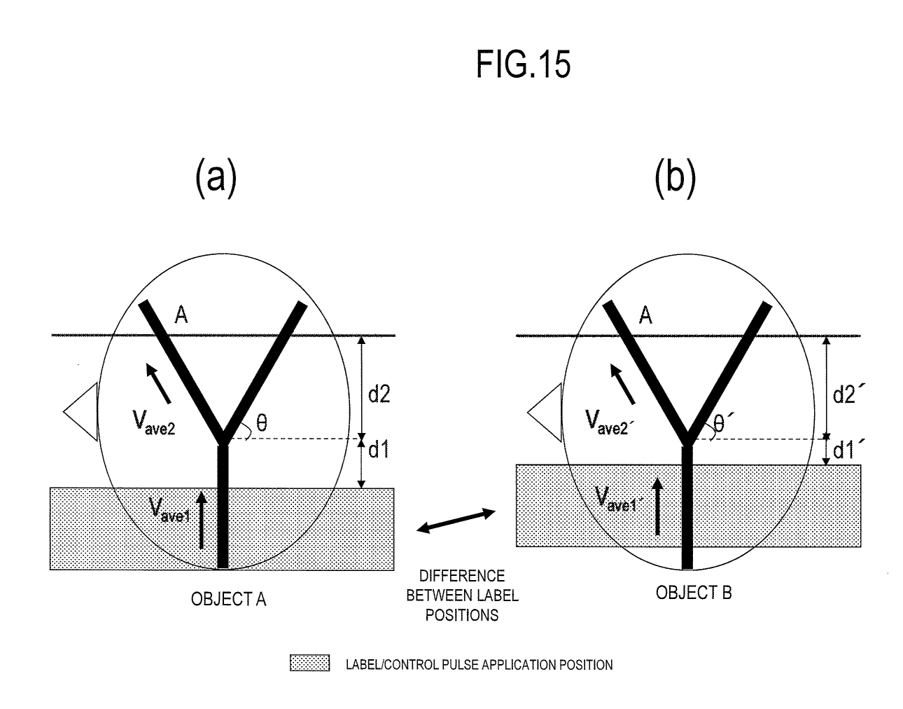

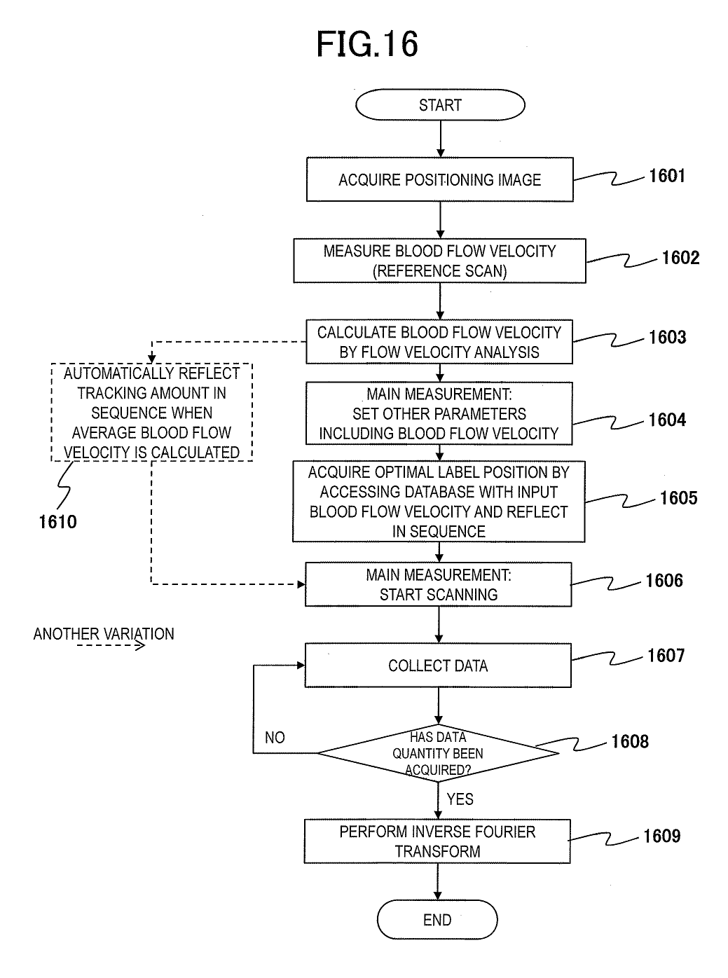

Although both of Equations (4) and (5) represent relationships between a blood flow velocity and optimal label or control positions in case of performing imaging at a predetermined PLD1000ms, different are objects and average blood flow velocities Vₐᵥₑ₁ and Vₐᵥₑ₂ to a branch point of a blood vessel. Therefore, a difference occurs between distances d1 and d1′ from application positions of label or control RF pulses optimal for a set PLD to the branch point as illustrated in FIGS. 15(a) and 15(b) (in which blood vessel running in the brain is simplified for easy understanding). There can be a difference between distances d2 and d2′ from the branch point to the point A in the imaging slice and between the branching angles θ. FIG. 16(a) represents Equation (4), and FIG. 16(b) represents Equation (5).

In other words, the optimal label or control positions are different depending on in case of performing imaging at a certain PLD set by a user (refer to the label positions A and B). The database 1084 stores such a relationship between a blood flow velocity and optimal label or control positions.

The sequence adjustment section 1082 adjusts application positions of RF pulses based on the optimal label or control positions acquired from the database 1084.

The scan control section 1083 performs control similarly to the description of Embodiment 2.

FIG. 16 illustrates operations the MRI apparatus and the control unit of the present embodiment.

Steps 1601 to 1604 are similar to Steps 1401 to 1404 of Embodiment 2.

In Step 1605, the entire control unit 108 accesses the database 1084 in order to acquire label or control positions optimal for an input blood flow velocity, and the sequence adjustment section 1082 reflects the optimal label or control positions to a sequence.

Steps 1606 to 1609 are similar to Steps 1406 to 1409 of Embodiment 2.

As a variation example illustrated in the dashed-line arrow of FIG. 16, it may be configured so that optimal label or control positions are automatically reflected to a sequence when a blood flow velocity is calculated in Step 1603 (Step 1610).

The present invention adjusts application positions of label or control pulses using a blood flow velocity, which can acquire highly reliable hemodynamic images in which the SNR was improved without being affected by the blood flow velocity at a PLD set by a user.

Although the case where either of the time distance or the space distance is adjusted is described in Embodiments 2 and 3, Embodiments 2 and 3 may be combined. That is, a PLD and application positions of label or control pulses may be controlled so as to spread out label- or control-processed blood protons in the entire region of interest and prevent longitudinal relaxation of the label- or control-processed blood protons from proceeding as possible.

### Embodiment 4

An MRI apparatus of Embodiment 4 is characterized by that a display/operation unit includes a function of displaying in color based on a threshold value of signal strength of a blood flow visualization image and changes the threshold value using a blood flow velocity. That is, Embodiment 4 uses the blood flow velocity as the threshold value for the color image display when an image of a region of interest is displayed (refer to the color display 207 of FIG. 2). It is noted that the blood flow velocity includes a blood flow velocity from a label or control processing region to an imaging region but is not limited to this.

Time until label- or control-processed blood reaches a region of interest varies depending on the object. Therefore, in case of performing imaging at the same PLD, a signal value of a result image varies depending on the object.

In case of displaying such a result image in color, the color display is generally performed based on a correspondence table (LUT) between signal values and colors (for example, a red color is changed to a blue one), and a color bar indicating the correspondence between the signal values and the colors is displayed together with an image to which the colors were assigned. For example, the color becomes red as the signal value increases, and the color becomes blue as the signal value decreases.

As described above, because the signal value varies depending on the blood flow velocity, whether or not there are many areas in an ischemia state and whether or not there are many areas displayed in blue due to the blood flow velocity cannot be determined in a case where a dynamic range (a threshold value of the signal value) to which a color is assigned, for example, when there are many areas in which the signal value is low (areas displayed in blue), which results in reducing an ability of perfusion visualization.

The present embodiment changes a threshold value of a signal value according to the blood flow velocity in order to improve the ability of perfusion visualization without depending on the blood flow velocity. Specifically, the blood flow velocity is used for optimizing color image display of a result image for each object. For example, the threshold value is reduced in a case where the blood flow velocity is low (the signal value is relatively low), and the threshold value is increased in a case where the blood flow velocity is high (the signal value is relatively high).

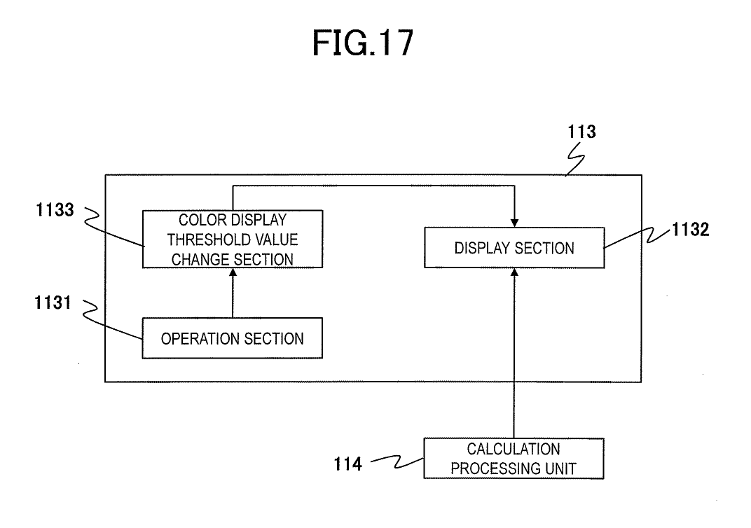

FIG. 17 is a functional block diagram in which the display/operation unit 113 of the present embodiment is mainly illustrated. The display/operation unit 113 includes an operation section 1131, a color display threshold value change section 1133, and a display section 1132. A measured blood flow velocity is input through the operation section 1131. The color display threshold value change section 1133 optimizes and sets a threshold value of a color bar based on the input blood flow velocity.

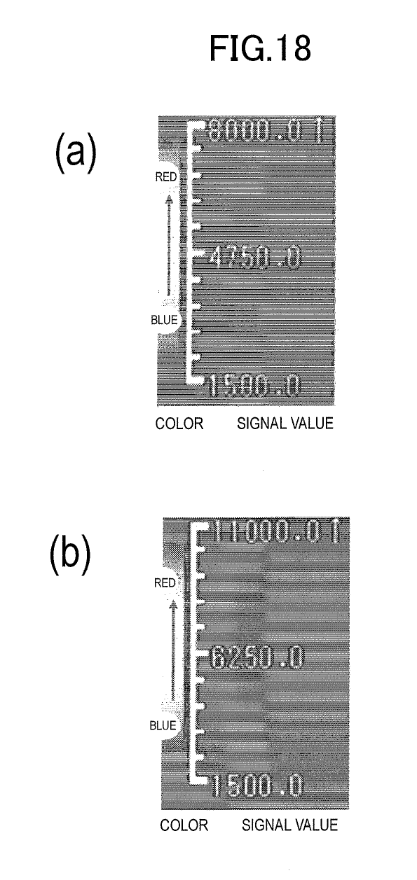

FIG. 18 shows an example of changing a threshold value according to the blood flow velocity. FIGS. 18(a) and 18(b) illustrates color bars in cases of the blood flow velocity 30 cm/s and lk 50 cm/s respectively, and the scales displayed on the color bars indicates signals values, in which red and blue areas are assigned to the upper and lower sides respectively.

For example, in a case where there are many areas displayed in blue because a blood flow velocity is low (30 cm/s) and the signal value is low, the upper limit value and the lower limit value of the signal value are set to 8,000 and 1,500 respectively in FIG. 18(a). By thus narrowing a dynamic range, a perfusion image can be displayed not only in the blue area but also in the red area even if signal strength is small. In other words, signal strength variation can be visualized in a more satisfactory manner.

On the other hand, for example, in a case where the signal value exceeds the color bar because the blood flow velocity is high (50 cm/s) and the signal value is high, the upper limit value and the lower limit value of the signal value are set to 11,000 and 1,500 respectively. By thus expanding the dynamic range, a perfusion image can be visualized more accurately in the color bar.

The display section 1132 displays hemodynamic images based on the set threshold value of the color bar.

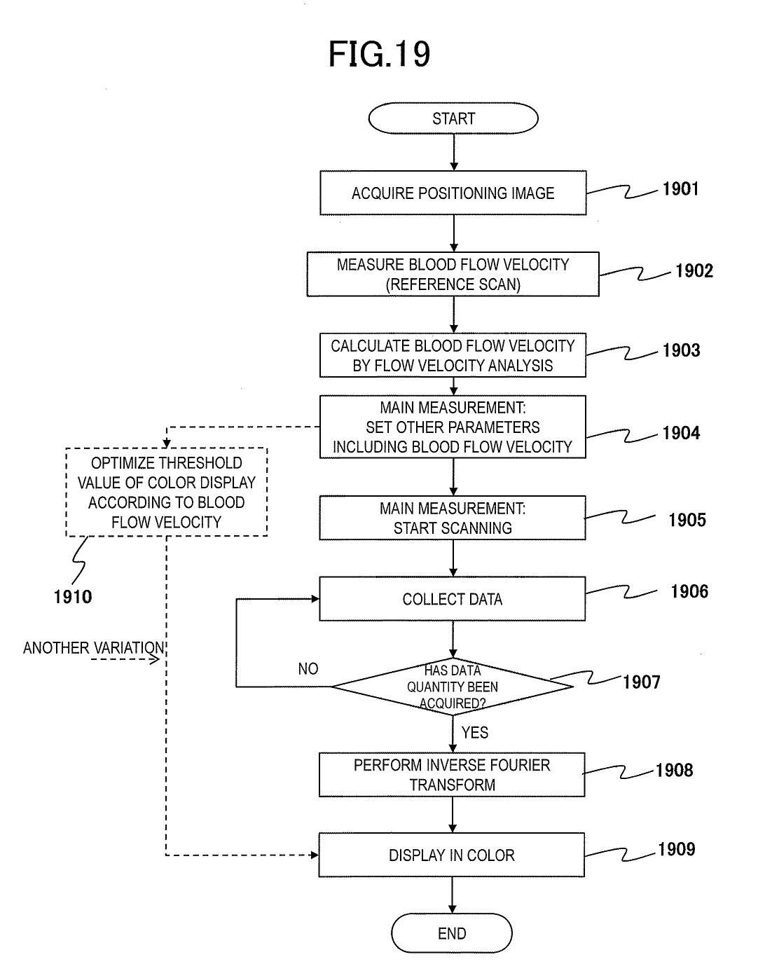

Processing procedures of the MRI apparatus, the control unit, and the display/operation unit of the present embodiment will be described using FIG. 19.

A positioning image for setting an imaging position is imaged (Step 1901).

Using the positioning image imaged in Step 1901, blood flow velocity measurement (reference scan) is performed on a region specified by an operator (Step 1902).

Based on the data acquired by the reference scan, a blood flow velocity graph is evaluated by the flow velocity analysis in order to calculate a blood flow velocity (Step 1903).

Set are scan parameters for main measurement input through the display/operation unit 113. At this time, the blood flow velocity evaluated in Step 1903 is also input as a scan parameter (Step 1904).

Pressing the start button starts scanning for main measurement (Step 1905). That is, performed are the label processing 2021, the hemodynamic imaging 2022, the control processing 2024, the hemodynamic imaging 2025, and the like of FIG. 2.

The measurement control unit 111 collects data (Step 1906).

The measurement control unit 111 judges whether or not an amount of data set by an operator in Step 1904 has been acquired and proceeds to Step 19006 in a case where the acquisition has not been completed or proceeds to Step 1908 in a case where the acquisition has been completed (Step 1907).

The calculation processing section 114 reconstructs two-dimensional or three-dimensional images by performing a Fourier transform on k-space data (Step 1908).

When the display/operation unit 113 displays a reconstructed image in color, the color display threshold value change section 1133 sets an optimal threshold value according to the blood flow velocity calculated in Step 1903 (Step 1910). Then, the display section 1132 performs color display using the threshold value (Step 1909).

It may be configured the optimal threshold value for color display is automatically reflected when the blood flow velocity is calculated in Step 1903 (not illustrated in the diagram).

The present embodiment can improve the reliability of the hemodynamic images displayed in color by changing a threshold value for the color display using the blood flow velocity.

Each the above-described Embodiments 1 to 4 may be performed alone, or one or more embodiments selected from Embodiments 1 to 4 may be combined and performed. In particular, it is desirable to combine Embodiments 1 and 4 in a case where Embodiments 2 and 3 are not performed.

### Embodiment 5

Although a blood flow velocity is acquired from the reference scan in Embodiments 1 to 4 (refer to 201 of FIG. 2), Embodiment 5 describes that it can be applied also to a case where the blood flow velocity is acquired from a database.

That is, an MRI apparatus of Embodiment 5 is characterized by that a control unit acquires a blood flow velocity by accessing the database that retains the standard blood flow velocity information. Although the step of the reference scan is not necessary in Embodiment 5, main measurement for acquiring hemodynamic images is similar to Embodiments 1 to 4.

FIG. 12 is a functional block diagram in which the entire control unit 108 of the present embodiment is mainly illustrated. In FIG. 12, the same functions as FIG. 1 are indicated with the same reference signs, and the descriptions thereof are omitted. The entire control unit 108 is characterized by including the database 1084 and the sequence adjustment section 1082.

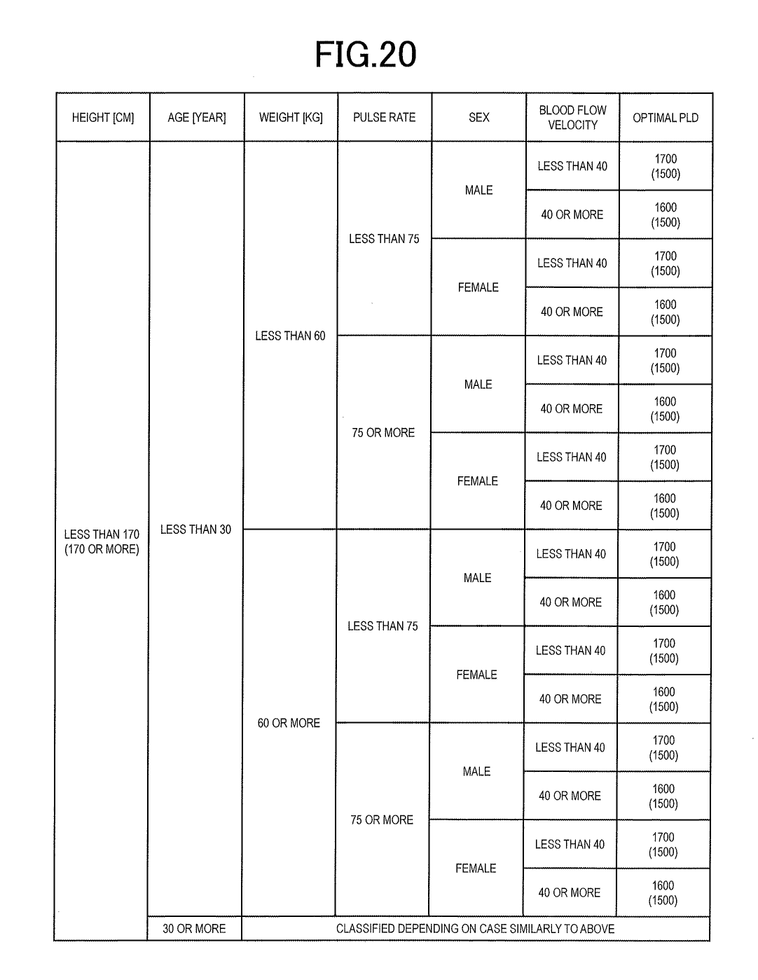

The database 1084 retains a general relationship between the height, age, weight, sex, pulse rate, and blood flow velocity of an object. An example of the database is indicated in FIG. 20. The information indicated in FIG. 20 is just an example, and more detailed information may be listed.

The entire control unit 108 accesses the database 1084 in order to acquire a blood flow velocity corresponding to object information input in the display/operation unit 113. The sequence adjustment section 1082 uses the acquired blood flow velocity in order to adjust a sequence as described in Embodiments 1 to 3. Although not illustrated in the diagram, the acquired blood flow velocity may be used for changing a threshold value for color display of Embodiment 4.

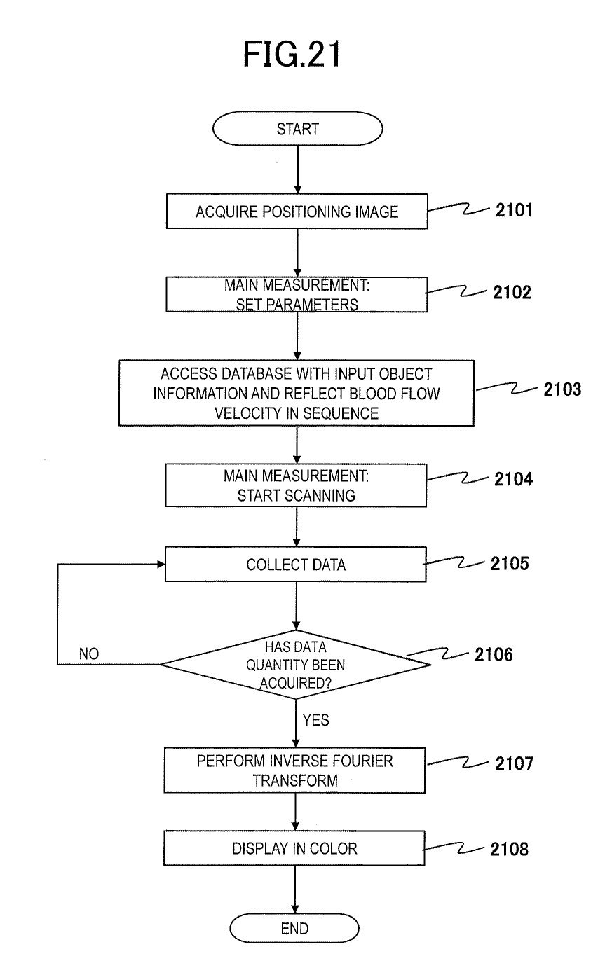

Processing procedures of the MRI apparatus, the control unit, and the display/operation unit of the present embodiment will be described using FIG. 21.

A positioning image for setting an imaging position is imaged (Step 2101).

Set are scan parameters for main measurement (Step 2102).

The entire control unit 108 accesses the database 1084 in order to acquire a blood flow velocity corresponding to the input object information. The sequence adjustment section 1082 uses the acquired blood flow velocity in order to adjust a sequence as described in Embodiments 1 to 3 (Step 2103).

Steps 2104 to 2107 are similar to those of Embodiments 1 to 4.

The color display in Step 2108 is similar to the color display 207 and can be processed similarly to Embodiment 4.

The same effect as Embodiments 1 to 4 can be acquired also in case of acquiring a blood flow velocity from a database in the present embodiment.

As described in each embodiment of the present invention, the present invention can be applied to any of two-dimensional and three-dimensional imaging methods in case of the acquisition method for non-contrast MR perfusion images, and a known pulse sequence may be adopted, including a spin echo-type echo planer method (SE-EPI), a fast spin echo method (FSE), a gradient echo-type echo planer method (GE-EPI), and the like.

The present invention can be applied to not only the head but also the entire body including the heart, kidney, liver, arms, and legs.

As described above in several embodiments of the present invention, the present invention can reduce the inaccuracy of non-contrast perfusion images, which can acquire hemodynamic images whose SNR is stably high. The reliability of hemodynamic images displayed in color can be also improved.

### REFERENCE SIGNS LIST

102: static magnetic field generating magnet, 103: gradient magnetic field coil, 109: gradient magnetic field power source, 104: transmission RF coil, 110: RF transmission unit, 105: reception RF coil, 106: signal detection unit, 111: measurement control unit (control unit), 108: entire control unit (control unit), 113: display/operation unit

1. A magnetic resonance imaging apparatus comprising:
a static magnetic field generating magnet; a high-frequency magnetic field generation unit; a gradient magnetic field generation unit; a reception unit that receives nuclear magnetic resonance signals; and a control unit that controls the high-frequency magnetic field generation unit; the gradient magnetic field generation unit; and the reception unit according to a predetermined pulse sequence,
wherein the pulse sequence includes sequences of applying a plurality of high-frequency pulses labeling a blood flow and imaging the blood flow, and
the control unit uses a blood flow velocity to control application positions of one or more high-frequency pulses from among the plurality of high-frequency pulses.

2. The magnetic resonance imaging apparatus according to claim 1,
wherein the control unit is provided with a label/control pulse tracking amount calculation section that calculates a correction amount of application positions of the second and subsequent high-frequency pulses compared with an application position of the first high-frequency pulse.

3. The magnetic resonance imaging apparatus according to claim 2,
wherein the control unit adjusts at least either of a frequency of high-frequency pulses or a gradient magnetic field application amount to control application positions of the second and subsequent high-frequency pulses.

4. The magnetic resonance imaging apparatus according to claim 1,
wherein the control unit further controls time between labeling the blood flow and starting the imaging and/or the application positions of high-frequency pulses for labeling the blood flow.

5. The magnetic resonance imaging apparatus according to claim 4,
wherein the time is between applying the last high-frequency pulse from among the plurality of high-frequency pulses and starting the imaging.

6. The magnetic resonance imaging apparatus according to claim 1, further comprising:
a display/operation unit that displays a blood flow visualization image in color based on a threshold value of the signal strength thereof,
wherein the display/operation unit changes the threshold value using a blood flow velocity.

7. The magnetic resonance imaging apparatus according to claim 1,
wherein the control unit performs measurement using a phase contrast method or accesses a database that retains standard blood flow velocity information in order to acquire the blood flow velocity.

8. The magnetic resonance imaging apparatus according to claim 1, further comprising:
the display/operation unit that displays a blood flow visualization image,
wherein the display/operation unit displays at least any of the blood flow velocity, the time between labeling the blood flow and starting the imaging, and the application positions of high-frequency pulses for labeling the blood flow.

9. The magnetic resonance imaging apparatus according to claim 1,
wherein the pulse sequence is selected from a group comprising PASL (Pulsed Arterial Spin Labeling), CASL (Continuous Arterial Spin Labeling), and pCASL (Pseudo-Continuous Arterial Spin Labeling).

10. A magnetic resonance imaging apparatus comprising:
a static magnetic field generating magnet; a high-frequency magnetic field generation unit; a gradient magnetic field generation unit; a reception unit that receives nuclear magnetic resonance signals; and a control unit that controls the high-frequency magnetic field generation unit; the gradient magnetic field generation unit; and the reception unit according to a predetermined pulse sequence,
wherein the pulse sequence includes sequences of applying high-frequency pulses labeling a blood flow and imaging the blood flow, and
the control unit uses a blood flow velocity to control time between labeling the blood flow and starting the imaging and/or the application positions of high-frequency pulses for labeling the blood flow.

11. A magnetic resonance imaging apparatus comprising:
a static magnetic field generating magnet; a high-frequency magnetic field generation unit; a gradient magnetic field generation unit; a reception unit that receives nuclear magnetic resonance signals; a control unit that controls the high-frequency magnetic field generation unit; the gradient magnetic field generation unit; and the reception unit according to a predetermined pulse sequence, and a display/operation unit that displays a blood flow visualization image,
wherein the pulse sequence includes sequences of applying high-frequency pulses labeling a blood flow and imaging the blood flow, and
the display/operation unit is provided with a function of displaying in color based on a threshold value of signal strength of the blood flow visualization image to change the threshold value using a blood flow velocity.

12. The magnetic resonance imaging apparatus according to claim 11,
wherein the display/operation unit further displays at least one of the blood flow velocity, the time between labeling the blood flow and starting the imaging, and the application positions of high-frequency pulses for labeling the blood flow.
